(19) United States
(12) Patent Application Publication
(10) Pub. No.: US 2014/0308224 A1
(43) Pub. Date: Oct. 16, 2014

(54) COSMETIC, DERMATOLOGICAL OR PHARMACEUTICAL COMPOSITIONS COMPRISING ISOSORBIDE DIESTERS AND UV FILTERS

(71) Applicants: Maurice Frederic Pilz, Frankfurt am Main (DE); Peter Klug, Grobostheim (DE); Franz-Xaver Scherl, Burgkirchen (DE)

(72) Inventors: Maurice Frederic Pilz, Frankfurt am Main (DE); Peter Klug, Grobostheim (DE); Franz-Xaver Scherl, Burgkirchen (DE)

(73) Assignee: CLARIANT INTERNATIONAL LTD., Muttenz (CH)

(21) Appl. No.: 14/237,076

(22) PCT Filed: Jul. 31, 2012
(86) PCT No.: PCT/EP2012/003249

§ 371 Date: Jul. 1, 2014

(30) Foreign Application Priority Data
Aug. 4, 2011 (DE) 10 2011 109 416.8

(51) Int. Cl. A61K 8/49; A61K 8/37; A61Q 17/04; A61K 8/34

(52) U.S. Cl. CPC A61K 8/4973; A61K 8/345; A61K 8/37; A61Q 17/04
USPC 424/60; 424/59

(57) ABSTRACT

Cosmetic, dermatological or pharmaceutical compositions comprising isosorbide diesters and UV filters

What are described are cosmetic, dermatological or pharmaceutical compositions comprising
a) one or more compounds of the formula (I)

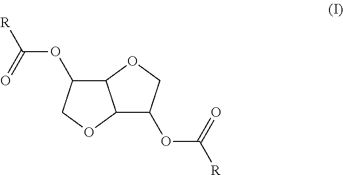

in which
R is a straight-chain or branched saturated alkyl group having 5 to 11 carbon atoms or a straight-chain or branched mono- or polyunsaturated alkenyl group having 5 to 11 carbon atoms, and
b) one or more organic or inorganic UV filters.

The present invention relates to cosmetic, dermatological or pharmaceutical compositions comprising one or more isosorbide diesters and one or more organic or inorganic UV filters.

Cosmetic, dermatological or pharmaceutical compositions comprising organic or inorganic UV filters are already known; however, they frequently have the disadvantage that the UV filters are poorly soluble and additionally are in particular poorly or unevenly distributed on the skin. As a consequence, the parts of the skin where there is only little or no UV filter are only poorly protected against UV radiation. This may result, for example, in premature aging of the skin and in an increased risk of skin cancer. There is therefore a need to distribute the UV filters as evenly as possible on the body sections to be protected. To overcome these disadvantages, use is therefore frequently made in the cosmetic, dermatological or pharmaceutical compositions of unwanted high amounts of solvents. However, high amounts of solvents may have the disadvantage that the skin dries out more and that additionally foreign substances penetrate the skin more easily. In addition, many solvents are based at least in part on synthetic raw materials.

It was therefore an object to provide cosmetic, dermatological or pharmaceutical compositions which comprise one or more organic or inorganic UV filters and do not have the disadvantages mentioned above, or where these disadvantages are at least reduced, and which are in particular distinguished by the advantages that they are based at least in part on renewable raw materials and dissolve or distribute UV filters relatively well and especially dissolve organic UV filters well, thus providing the precondition that, when the compositions are applied, the UV filters can be distributed relatively evenly on the skin.

Surprisingly, it has now been found that this object is achieved by cosmetic, dermatological or pharmaceutical compositions comprising

a) one or more compounds of the formula (I)

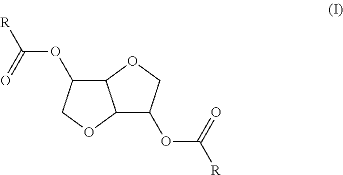

in which
R is a straight-chain or branched saturated alkyl group having 5 to 11, preferably 7 to 9 and particularly preferably 7 carbon atoms or a straight-chain or branched mono- or polyunsaturated alkenyl group having 5 to 11, preferably 7 to 9 and particularly preferably 7 carbon atoms, and
   b) one or more organic or inorganic UV filters.

Accordingly, the invention provides cosmetic, dermatological or pharmaceutical compositions, preferably sun protection formulations, comprising

a) one or more compounds of the formula (I)

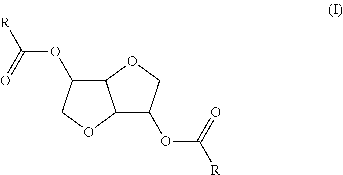

in which
R is a straight-chain or branched saturated alkyl group having 5 to 11, preferably 7 to 9 and particularly preferably 7 carbon atoms or a straight-chain or branched mono- or polyunsaturated alkenyl group having 5 to 11, preferably 7 to 9 and particularly preferably 7 carbon atoms, and
   b) one or more organic or inorganic UV filters.

By virtue of the presence of the compounds of the formula (I), the compositions according to the invention are based on renewable raw materials. Since the compounds of the formula (I) additionally dissolve the organic UV filters particularly well, the use concentration of solvents customarily employed may be reduced.

Compositions, for example cosmetic, dermatological or pharmaceutical compositions, comprising esters based on renewable raw materials are already known.

WO 2010/108738 A2 (Evonik) describes formulations which are used to clean and care for human or animal body parts and comprise sorbitancarboxylic esters, where the carboxylic acid portion of the sorbitancarboxylic ester is derived from a carboxylic acid containing 6 to 10 carbon atoms and the sorbitancarboxylic esters have a hydroxyl value of more than 350, and the use of the sorbitancarboxylic esters mentioned as viscosity regulators, care active ingredient, foam booster or solubilizer in cleaning or care formulations.

DE 10 2009 022 444 (Clariant) describes liquid compositions comprising sorbitan monocaprylate and other antimicrobially active compounds such as, for example, certain organic acids and their salts, certain formeldehyde donors, certain isothiazolinones (e.g. methylchloroisothiazolinone), certain paraben esters or certain pyridones and their salts, and also their use for preserving cosmetic, dermatological or pharmaceutical products.

DE 10 2009 022 445 (Clariant) discloses liquid compositions comprising sorbitan monocaprylate and alcohol and their use for preserving cosmetic, dermatological or pharmaceutical products.

JP 8173787 (A) (Lion) describes a composition comprising a surfactant comprising a fatty ester of dehydrated sorbitol, and the use as oil-in-water emulsifier and as cleaner base. The compositions may comprise mono- or diesters of caprylic and/or caprinic acid with a polyol selected from the group consisting of 1,5-sorbitan, 1,4-sorbitan and isosorbide.

JP 8187070 (A) (Lion) discloses a mixture of fatty acid monoesters of C₈-C₁₈ fatty acids and at least one polyol selected from sorbitol, 1,5-sorbitan, 1,4-sorbitan and isosorbide and fatty acid diesters of these fatty acids and polyols in a weight ratio of monoester:diester of 33:7 to 9:1 as antimicrobially active compound against bacteria for food or beverages. However, food or beverages typically do not comprise any UV filters.

Compounds of components a) and b) of the compositions according to the invention are commercially available or can be produced by methods known to the person skilled in the art. For example, the compounds of the formula (I) can be prepared by esterification of isosorbide by customary methods known to the person skilled in the art, with both isosorbide for its part and also the acid components used for esterification once more being commercially available.

Preferably, the radical R in the one or more compounds of the formula (I) is a straight-chain saturated alkyl radical having 7 to 9 carbon atoms.

Particularly preferably, the radical R in the one or more compounds of the formula (I) is a straight-chain saturated alkyl radical having 7 carbon atoms.

The organic UV filters of component b) of the compositions according to the invention are preferably selected from the group consisting of 4-aminobenzoic acid, 3-(4′-trimethylammonium)benzylideneboran-2-one methyl sulfate, camphorbenzalkonium methosulfate, 3,3,5-trimethylcyclohexyl salicylate, 2-hydroxy-4-methoxybenzophenone, 2-phenylbenzimidazole-5-sulfonic acid and its potassium, sodium and triethanolamine salts, 3,3′-(1,4-phenylenedimethine)bis(7,7-dimethyl-2-oxobicyclo[2.2.1]heptane-1-methanesulfonic acid) and its salts, 1-(4-tert-butylphenyl)-3-(4-methoxyphenyl)propane-1,3-dione, 3-(4′-sulfo)benzylidenebornan-2-one and its salts, 2-ethylhexyl 2-cyano-3,3-diphenylacrylate, polymers of N-[2(and 4)-(2-oxoborn-3-ylidenemethyl)benzyl]acrylamide, 2-ethylhexyl 4-methoxycinnamate, ethoxylated ethyl 4-aminobenzoate, isoamyl 4-methoxycinnamate, 2,4,6-tris[p-(2-ethylhexyloxycarbonyl)anilino]-1,3,5-triazine, 2-(2H-benzotriazol-2-yl)-4-methyl-6-(2-methyl-3-(1,3,3,3-tetramethyl-1-(trimethylsilyloxy)disiloxanyl)propyl)phenol, bis(2-ethylhexyl) 4,4′-[(6-[4-((1,1-dimethylethypaminocarbonyl)phenylamino]-1,3,5-triazin-2,4-yl)diimino]bisbenzoate, benzophenone-3, benzophenone-4 (acid), 3-(4′-methylbenzylidene)-D,L-camphor, 3-benzylidenecamphor, 2-ethylhexyl salicylate, 2-ethylhexyl 4-dimethylaminobenzoate, hydroxy-4-methoxybenzophenone-5-sulfonic acid (sulfisobenzone) and the sodium salt, 4-isopropylbenzyl salicylate, N,N,N-trimethyl-4-(2-oxoborn-3-ylidenemethyl)anilium methyl sulfate, homosalate (INN), oxybenzone (INN), 2-phenylbenzimidazole-5-sulfonic acid and its sodium, potassium and triethanolamine salts, octylmethoxycinnamic acid, isopentyl-4-methoxycinnamic acid, isoamyl-p-methoxycinnamic acid, 2,4,6-trianilino(p-carbo-2′-ethylhexyl-1′-oxy)-1,3,5-triazine (octyltriazone) phenol, 2-2(2H-benzotriazol-2-yl)-4-methyl-6-(2-methyl-3-(1,3,3,3-tetramethyl-1-(trimethylsilyl)oxy)disiloxanyl)propyl(drometrizoletrisiloxane)benzoic acid, 4,4-((6-(((1,1-dimethylethyl)amino)carbonyl)phenyl)amino)-1,3,5-triazine-2,4-diyl)diimino)bis,bis(2-ethylhexyl) ester)benzoic acid, 4,4-((6-(((1,1-dimethylethyl)amino)carbonyl)phenyl)amino)-1,3,5-triazine-2,4-diyl)diimino)bis,bis(2-ethylhexyl)ester), 3-(4′-methylbenzylidene)-D,L-camphor (4-methylbenzylidenecamphor), benzylidenecamphorsulfonic acid, octocrylene, polyacrylamidomethylbenzylidenecamphor, 2-ethylhexyl salicylate (octylsalicylate), ethyl-2-hexyl 4-dimethylaminobenzoate (octyldimethyl PABA), PEG-25 PABA, 2-hydroxy-4-methoxybenzophenone-5-sulfonic acid (benzophenone-5) and the Na salt, 2,2′-methylenebis-6-(2H-benzotriazol-2-yl)-4-(tetramethylbutyl)-1,1,3,3-phenol, sodium salt of 2-2′-bis(1,4-phenylene)-1H-benzimidazole-4,6-disulfonic acid, (1,3,5)-triazine-2,4-bis((4-(2-ethylhexyloxy)-2-hydroxy)phenyl)-6-(4-methoxyphenyl), 2-ethylhexyl 2-cyano-3,3-diphenyl-2-propenoate, glyceryl octanoate, di-p-methoxycinnamic acid, p-aminobenzoic acid and esters thereof, 4-tert-butyl-4′-methoxydibenzoylmethane, 4-(2-β-glucopyranoxy)propoxy-2-hydroxybenzophenone, octyl salicylate, methyl-2,5-diisopropylcinnamic acid, cinoxate, dihydroxydimethoxybenzophenone, disodium salt of 2,2′-dihydroxy-4,4′-dimethoxy-5,5′-disulfobenzophenone, dihydroxybenzophenone, 1,3,4-dimethoxyphenyl-4,4-dimethyl-1,3-pentanedione, 2-ethylhexyl dimethoxybenzylidenedioxoimidazolidinepropionate, methylenebisbenzotriazolyl tetramethylbutylphenol, phenyl dibenzimidazoletetrasulfonate, bisethylhexyloxyphenol methoxyphenol triazine, tetrahydroxybenzophenones, terephthalylidenedicamphorsulfonic acid, 2,4,6-tris[4,2-ethylhexyloxycarbonyl)anilino]-1,3,5-triazine, methylbis(trimethylsiloxy)silylisopentyltrimethoxycinnamic acid, amyl p-dimethylaminobenzoate, amyl p-dimethylaminobenzoate, 2-ethylhexyl p-dimethylaminobenzoate, isopropyl-p-methoxycinnamic acid/diisopropylcinnamic acid esters, 2-ethylhexyl-p-methoxycinnamic acid, 2-hydroxy-4-methoxybenzophenone, 2-hydroxy-4-methoxybenzophenone-5-sulfonic acid and the trihydrate, and also 2-hydroxy-4-methoxybenzophenone-5-sulfonate sodium salt and phenylbenzimidazolesulfonic acid.

The inorganic UV filters of component b) of the compositions according to the invention are preferably selected from the group consisting of microfine titanium dioxide, mica/titanium oxide, iron oxide, mica/iron oxide, zinc oxide, silicon oxide, ultramarine blue and chromium oxide.

Preferably, the one or more UV filters of component b) are selected from among organic UV filters.

The one or more organic UV filters of component b) are preferably selected from the group consisting of butyl methoxydibenzoylmethane (BMDBM), octocrylene, ethylhexyl methoxycinnamate (EMC), ethylhexyl triazone (EHT), diethylhexyl butamido triazone, bisethylhexyloxyphenol methoxyphenyl triazine, 4-methylbenzylidene camphor, benzyl salicylate, ethylhexyl salicylate, terephthalydene dicamphor sulfonic acid, ethylhexyl methoxycinnamate and disodium octanyl dibenzimidazole tetrasulfonate.

Particularly preferably, the one or more organic UV filters of component b) are selected from the group consisting of butyl methoxydibenzoylmethane, octocrylene, ethylhexyl methoxycinnamate and ethylhexyl triazone.

Especially preferably, the organic UV filter of component b) is butyl methoxydibenzoylmethane.

Most preferably, the radical R in the one or more compounds of the formula (I) is a straight-chain saturated alkyl radical having 7 carbon atoms, and the UV filter of component b) is butyl methoxydibenzoylmethane.

In a further preferred embodiment of the invention, the compositions according to the invention comprise one or more isosorbide monoesters of the formula (II)

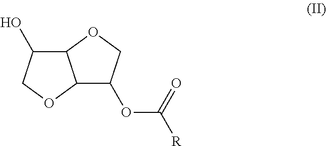

where R has the meaning given for formula (I) and where the isosorbide monoester is preferably isosorbide monocaprylate.

In a preferred embodiment of this embodiment of the invention, the compositions according to the invention comprise one or more compounds of the formula (I) and additionally
II) from 0.001 to 0.2, preferably from 0.01 to 0.15 and particularly preferably from 0.05 to 0.13 part by weight of the one or more isosorbide monoesters of the formula (II), where the isosorbide monoester is preferably isosorbide monocaprylate,
   in each case based on 1.0 part by weight of the one or more compounds of the formula (I) and preferably based on 1.0 part by weight of isosorbide dicaprylate. In a further preferred embodiment of the invention, the compositions according to the invention comprise either no carboxylic acid RCOOH or up to 0.1, preferably from 0.0001 to 0.05 and particularly preferably from 0.001 to 0.01 part by weight of carboxylic acid RCOOH, where R has the meaning given for formula (I) and where the carboxylic acid is preferably caprylic acid, based on 1.0 part by weight of the one or more compounds of the formula (I) and preferably based on 1.0 part by weight of isosorbide dicaprylate.

In a further preferred embodiment of the invention, the compositions according to the invention additionally comprise one or more sorbitan esters of sorbitan and carboxylic acids R^{a}COOH, preferably selected from sorbitan esters of 1,4- and/or 1,5-sorbitan and carboxylic acids R^{a}COOH, where R^{a} is a straight-chain or branched saturated alkyl group having 5 to 11, preferably 7 to 9 and particularly preferably 7 carbon atoms or a straight-chain or branched mono- or polyunsaturated alkenyl group having 5 to 11, preferably 7 to 9 and particularly preferably 7 carbon atoms, and where the weight ratio of the one or more compounds of the formula (I) to the one or more sorbitan esters just mentioned is from 70:30 to 100:0, preferably from 80:20 to 100:0, particularly preferably from 90:10 to 100:0 and especially preferably from 95:5 to 100:0. The stated weight ratio of “100:0” means that in this preferred embodiment of the invention, the compositions according to the invention do not need to comprise any sorbitan ester.

From among the compositions according to the invention just mentioned, preference is given to those in which the one or more sorbitan esters of sorbitan and carboxylic acids R^{a}COOH are selected from sorbitan esters of sorbitan and caprylic acid and preferably selected from sorbitan esterns of 1,4- and/or 1,5-sorbitan and caprylic acid and the sorbitan ester is particularly preferably sorbitan dicaprylate.

In these compositions, the hydroxyl value of the mixture of the one or more compounds of the formula (I) and the one or more (if present) sorbitan esters of sorbitan and carboxylic acids R^{a}COOH is preferably smaller than or equal to 320, particularly preferably smaller than or equal to 285, especially preferably smaller than or equal to 245 and most preferably smaller than or equal to 225.

In a further preferred embodiment of the invention, the compositions according to the invention comprise, in addition to the one or more compounds of the formula (I), one or more compounds selected from the group consisting of sorbitol, sorbitol esters (sorbitol esters can be mono-, di-, tri-, tetra-, penta- and/or hexaesters), sorbitan, sorbitan esters (sorbitan esters can be mono-, di-, tri- and/or tetraesters), isosorbide, isosorbide monoesters and carboxylic acids. “Sorbitan” can be, for example, 1,4- or 1,5-sorbitan. Both the carboxylic acids themselves and the carboxylic acids on which the acid components of the esters mentioned are based correspond to the formula RCOOH in which R has the meaning given for formula (I) and is preferably a straight-chain saturated alkyl radical having 7 carbon atoms, i.e. the carboxylic acid RCOOH is preferably caprylic acid. In the preferred embodiment of the invention described herein, the hydroxyl value of the mixture of the one or more compounds of the formula (I) and the one or more compounds selected from the group consisting of sorbitol, sorbitol esters, sorbitan, sorbitan esters, isosorbide, isosorbide monoesters and carboxylic acids is smaller than or equal to 250, preferably smaller than or equal to 200, particularly preferably smaller than or equal to 150 and especially preferably smaller than or equal to 100.

In a further preferred embodiment of the invention, the compositions according to the invention do not comprise any compounds selected from the group consisting of sorbitol and sorbitol esters. However, if the cosmetic, dermatological or pharmaceutical compositions according to the invention do comprise one or more compounds selected from the group consisting of sorbitol and sorbitol esters (where the carboxylic acid on which the acid component of these esters is based is preferably caprylic acid), these compounds together are preferably present in the cosmetic, dermatological or pharmaceutical compositions according to the invention in an amount smaller than or equal to 0.1% by weight, particularly preferably in an amount smaller than or equal to 0.06% by weight, especially preferably in an amount smaller than or equal to 0.02% by weight and most preferably in an amount smaller than or equal to 0.01% by weight, the stated % by weight in each case being based on the total weight of the finished composition according to the invention.

In a further preferred embodiment of the invention, the compositions according to the invention do not comprise any compounds selected from the group consisting of sorbitan and sorbitan esters. However, if the cosmetic, dermatological or pharmaceutical compositions according to the invention do comprise one or more compounds selected from the group consisting of sorbitan and sorbitan esters (where the carboxylic acid on which the acid component of these esters is based is preferably caprylic acid), these compounds together are preferably present in the cosmetic, dermatological or pharmaceutical compositions according to the invention in an amount smaller than or equal to 0.4% by weight, particularly preferably in an amount smaller than or equal to 0.2% by weight, especially preferably in an amount smaller than or equal to 0.1% by weight and most preferably in an amount smaller than or equal to 0.02% by weight, the stated % by weight in each case being based on the total weight of the finished composition according to the invention.

The hydroxyl value of a substance is to be understood as meaning that amount of KOH in mg equivalent to the amount of acetic acid bound during the acetylation of 1 g of substance.

Suitable determination methods for determining the hydroxyl value are, for example, DGF C-V 17 a (53), Ph. Eur. 2.5.3 Method A and DIN 53240.

In the context of the present invention, the hydroxyl values are determined analogously to DIN 53240-2. Here, the following procedure is adopted: 1 g, accurate to 0.1 mg, of the homogenized sample to be measured is weighed out. 20.00 ml of acetylation mixture (acetylation mixture: 50 ml of acetic anhydride are stirred into 1 l of pyridine) are added. The sample is dissolved completely in the acetylation mixture, if required with stirring and heating. 5 ml of catalyst solution (catalyst solution: 2 g of 4-dimethylaminopyridine are dissolved in 100 ml of pyridine) are added. The reaction vessel is closed and placed into the water bath, preheated to 55° C., for 10 minutes, with mixing. 10 ml of fully deionized water are then added to the reaction solution, the reaction vessel is closed again and the mixture is once more allowed to react in the water bath with shaking for 10 minutes. The sample is cooled to room temperature (25° C.). 50 ml of 2-propanol and 2 drops of phenolphthalein are then added. This solution is titrated with aqueous sodium hydroxide solution (aqueous sodium hydroxide solution c=0.5 mol/l) (Va). Under identical conditions, but without any sample added, the efficacy of the acetylation mixture is determined (Vb).

From the aqueous sodium hydroxide solution consumed in the determination of the efficacy and the titration of the sample, the hydroxyl value is calculated using the following formula:

$$OHV = \frac{( Vb - Va ) \cdot c \cdot t \cdot M}{E}$$

OHV=hydroxyl value in mg KOH/g substance
Va=aqueous sodium hydroxide solution consumed in ml during the titration of the sample
Vb=aqueous sodium hydroxide solution consumed in ml during the titration of the efficacy
c=molar concentration of the aqueous sodium hydroxide solution in mol/1
t=titer of the aqueous sodium hydroxide solution
M=molar mass of KOH=56.11 g/mol
E=sample weighed out in g

(Vb−Va) is that amount of aqueous sodium hydroxide solution used in ml, which is equivalent to the amount of acetic acid bound during the above-described acetylation of the sample to be measured.

Hereinbelow, the method just described for determining the hydroxyl value is referred to as “method OHV-A”.

The cosmetic, dermatological or pharmaceutical compositions according to the invention comprise the one or more compounds of component a) preferably in amounts of from 0.01 to 10.0% by weight, particularly preferably in amounts of from 0.1 to 5.0% by weight, especially preferably in amounts of from 0.2 to 3.0% by weight and most preferably in amounts of from 0.5 to 2.0% by weight and the one or more substances of component b) preferably in amounts of from 0.01 to 40.0% by weight, particularly preferably in amounts of from 0.2 to 30.0% by weight, especially preferably in amounts of from 0.5 to 15.0% by weight and most preferably in amounts of from 1.0 to 10.0% by weight, in each case based on the total weight of the composition according to the invention.

In a further preferred embodiment of the invention, the cosmetic, dermatological or pharmaceutical compositions according to the invention have viscosities preferably in the range from 50 to 200 000 mPa·s, particularly preferably in the range from 500 to 100 000 mPa·s, especially preferably in the range from 2 000 to 50 000 mPa·s and most preferably in the range from 5 000 to 30 000 mPa·s (20° C., Brookfield RVT, RV spindle set at 20 revolutions per minute).

In a further preferred embodiment of the invention, the cosmetic, dermatological or pharmaceutical compositions according to the invention are present in the form of fluids, gels, foams, sprays, lotions or creams.

The cosmetic, dermatological or pharmaceutical compositions according to the invention are preferably formulated on an aqueous or aqueous-alcoholic basis or are present as solutions, emulsions or dispersions. Particularly preferably, they are present as emulsions, and especially preferably they are present as oil-in-water emulsions.

All mono- or polyhydric alcohols are suitable for the cosmetic, dermatological or pharmaceutical compositions according to the invention on an aqueous-alcoholic or else alcoholic basis. Preference is given to using alcohols having 1 to 4 carbon atoms, such as ethanol, propanol, isopropanol, n-butanol, isobutanol, t-butanol or glycerol, and also alkylene glycols, in particular propylene glycol, butylene glycol or hexylene glycol, and mixtures of said alcohols. Further preferred alcohols are polyethylene glycols with a relative molecular mass below 2 000. Particular preference is given to using ethanol or isopropanol.

The cosmetic, dermatological or pharmaceutical compositions according to the invention may comprise one or more oils.

Advantageously, the oils may be selected from the groups of the triglycerides, natural and synthetic fatty substances, preferably esters of fatty acids with alcohols having a low carbon number, for example with methanol, isopropanol, propylene glycol or glycerol, or esters of fatty alcohols with alkanoic acids having a low carbon number or with fatty acids or from the group of the alkyl benzoates, and also natural or synthetic hydrocarbon oils.

Suitable are triglycerides of straight-chain or branched saturated or unsaturated, optionally hydroxylated C₈-C₃₀-fatty acids, in particular vegetable oils such as sunflower oil, corn oil, soybean oil, rice oil, jojoba oil, babusscu oil, pumpkin oil, grapeseed oil, sesame oil, walnut oil, apricot oil, orange oil, wheatgerm oil, peach kernel oil, macadamia oil, avocado oil, sweet almond oil, lady's smock oil, castor oil, olive oil, peanut oil, rapeseed oil and coconut oil, and also synthetic triglyceride oils, for example the commercial product Myritol® 318. Hydrogenated triglycerides are also suitable. Oils of animal origin, for example beef tallow, perhydrosqualene, lanolin, can also be used.

A further class of preferred oily substances comprises the benzoic acid esters of linear or branched C₈₋₂₂-alkanols, for example the commercial products Finsolv® SB (isostearyl benzoate), Finsolv® TN (C₁₂-C₁₅-alkyl benzoate) and Finsolv® EB (ethylhexyl benzoate).

A further class of preferred oily substances comprises the dialkyl ethers having in total 12 to 36 carbon atoms, in particular having 12 to 24 carbon atoms, such as, for example, di-n-octyl ether (Cetiol® OE), di-n-nonyl ether, di-n-decyl ether, di-n-undecyl ether, di-n-dodecyl ether, n-hexyl n-octyl ether, n-octyl n-decyl ether, n-decyl n-undecyl ether, n-undecyl n-dodecyl ether and n-hexyl n-undecyl ether, di-3-ethyldecyl ether, tert-butyl n-octyl ether, isopentyl n-octyl ether and 2-methylpentyl n-octyl ether, and also di-tert-butyl ether and diisopentyl ether.

Branched saturated or unsaturated fatty alcohols having 6-30 carbon atoms, e.g. isostearyl alcohol, and Guerbet alcohols, are likewise suitable.

A further class of preferred oily substances comprises hydroxycarboxylic acid alkyl esters. Preferred hydroxycarboxylic acid alkyl esters are full esters of glycolic acid, lactic acid, malic acid, tartaric acid or citric acid. Further esters of hydroxycarboxylic acids which are suitable in principle are esters of β-hydroxypropionicacid, of tartronic acid, of D-gluconic acid, sugar acid, mucic acid or glucuronic acid. Suitable alcohol components of these esters are primary straight-chain or branched aliphatic alcohols having 8 to 22 carbon atoms. Here, the esters of C₁₂-C₁₅-fatty alcohols are particularly preferred. Esters of this type are commercially available, e.g. under the trade name Cosmacol® from EniChem, Augusta Industriale.

A further class of preferred oily substances comprises dicarboxylic acid esters of straight-chain or branched C₂-C₁₀-alkanols, such as di-n-butyl adipate (Cetiol® B), di(2-ethylhexyl)adipate and di(2-ethylhexyl) succinate, and also diol esters, such as ethylene glycol dioleate, ethylene glycol diisotridecanoate, propylene glycol di(2-ethylhexanoate), propylene glycol diisostearate, propylene glycol dipelargonate, butanediol diisostearate and neopentyl glycol dicaprylate, and also diisotridecyl azelate.

Likewise preferred oily substances are symmetrical, asymmetrical or cyclic esters of carbonic acid with fatty alcohols, glycerol carbonate or dicaprylyl carbonate (Cetiol® CC).

A further class of preferred oily substances comprises the esters of dimers of unsaturated C₁₂-C₂₂-fatty acids (dimer fatty acids) with monohydric straight-chain, branched or cyclic C₂-C₁₈-alkanols or with polyhydric straight-chain or branched C₂-C₆-alkanols.

A further class of preferred oily substances comprises hydrocarbon oils, for example those with straight-chain or branched, saturated or unsaturated C₇-C₄₀-carbon chains, for example Vaseline, dodecane, isododecane, cholesterol, lanolin, synthetic hydrocarbons such as polyolefins, in particular polyisobutene, hydrogenated polyisobutene, polydecane, and hexadecane, isohexadecane, paraffin oils, isoparaffin oils, for example the commercial products of the Permethyl® series, squalane, squalene, and alicyclic hydrocarbons, for example the commercial product 1,3-di(2-ethylhexyl)cyclohexane (Cetiol® S), ozokerite, and ceresine.

Also suitable are silicone oils and silicone waxes, preferably dimethylpolysiloxanes and cyclomethicones, polydialkylsiloxanes R₃SiO(R₂SiO)ₓSiR₃, where R is methyl or ethyl, particularly preferably methyl, and x is a number from 2 to 500, for example the dimethicones available under the trade names VICASIL (General Electric Company), DOW CORNING 200, DOW CORNING 225, DOW CORNING 200 (Dow Corning Corporation), and also the dimethicones available under SilCare® Silicone 41 M65, SilCare® Silicone 41 M70, SilCare® Silicone 41 M80 (Clariant), stearyldimethylpolysiloxane, C₂₀-C₂₄-alkyldimethylpolysiloxane, C₂₄-C₂₈-alkyldimethylpolysiloxane; but also the methicones available under SilCare® Silicone 41M40, SilCare® Silicone 41M50 (Clariant), furthermore trimethylsiloxysilicates [(CH₂)₃SiO)_{1/2}]ₓ[SiO₂]_{y}, where x is a number from 1 to 500 and y is a number from 1 to 500, dimethiconols R₃SiO[R₂SiO]ₓSiR₂OH and HOR₂SiO[R₂SiO]ₓSiR₂OH, where R is methyl or ethyl and x is a number up to 500, polyalkylarylsiloxanes, for example the polymethylphenylsiloxanes available under the trade names SF 1075 METHYLPHENYL FLUID (General Electric Company) and 556 COSMETIC GRADE PHENYL TRIMETHICONE FLUID (Dow Corning Corporation), polydiarylsiloxanes, silicone resins, cyclic silicones and amino-, fatty-acid-, alcohol-, polyether-, epoxy-, fluorine- and/or alkyl-modified silicone compounds, and also polyether siloxane copolymers.

As further auxiliaries and additives, the cosmetic, dermatological or pharmaceutical compositions according to the invention may comprise, for example, waxes, emulsifiers, coemulsifiers, solubilizers, electrolytes, hydroxy acids, stabilizers, cationic polymers, film formers, thickeners, gelling agents, superfattening agents, refattening agents, antimicrobially active compounds, biogenic active compounds, astringents, deodorizing substances, antioxidants, humectants, solvents, colorants, pearlizing agents, fragrances, opacifiers and/or silicones.

The cosmetic, dermatological or pharmaceutical compositions according to the invention may comprise waxes, for example paraffin waxes, microwaxes and ozokerites, beeswax and its partial fractions, and also beeswax derivatives, waxes from the group of homopolymeric polyethylenes or copolymers of α-olefins, and natural waxes such as rice wax, candelilla wax, carnauba wax, Japan wax or shellac wax.

Emulsifiers, coemulsifiers and solubilizers which can be used are nonionic, anionic, cationic or amphoteric surface-active compounds.

Suitable nonionic surface-active compounds are preferably: addition products of from 1 to 30 mol of ethylene oxide and/or 1 to 5 mol of propylene oxide onto linear fatty alcohols having 8 to 22 carbon atoms, onto fatty acids having 12 to 22 carbon atoms, onto alkylphenols having 8 to 15 carbon atoms in the alkyl group and onto sorbitan or sorbitol esters; (C₁₂-C₁₈)-fatty acid mono- and diesters of addition products of from 1 to 30 mol of ethylene oxide onto glycerol; glycerol mono- and diesters and sorbitan mono- and diesters of saturated and unsaturated fatty acids having 14 to 22 carbon atoms and optionally ethylene oxide addition products thereof; addition products of from 15 to 60 mol of ethylene oxide onto castor oil and/or hydrogenated castor oil; polyol and in particular polyglycerol esters, such as, for example, polyglycerol polyricinoleate and polyglycerol poly-12-hydroxystearate. Ethoxylated fatty amines, fatty acid amides, fatty acid alkanolamides and mixtures of compounds of two or more of these substance classes are likewise preferably suitable.

Suitable ionogenic coemulsifiers are, for example, anionic emulsifiers, such as mono-, di- or triphosphoric acid esters, soaps (e.g. sodium stearate), fatty alcohol sulfates, but also cationic emulsifiers such as mono-, di- and trialkyl quats and polymeric derivatives thereof.

Available amphoteric emulsifiers are preferably alkylaminoalkylcarboxylic acids, betaines, sulfobetaines and imidazoline derivatives.

Particular preference is given to using fatty alcohol ethoxylates selected from the group consisting of ethoxylated stearyl alcohols, isostearyl alcohols, cetyl alcohols, isocetyl alcohols, oleyl alcohols, lauryl alcohols, isolauryl alcohols and cetylstearyl alcohols, in particular polyethylene glycol(13) stearyl ether, polyethylene glycol(14) stearyl ether, polyethylene glycol(15) stearyl ether, polyethylene glycol(16) stearyl ether, polyethylene glycol(17) stearyl ether, polyethylene glycol(18) stearyl ether, polyethylene glycol(19) stearyl ether, polyethylene glycol(20) stearyl ether, polyethylene glycol(12) isostearyl ether, polyethylene glycol(13) isostearyl ether, polyethylene glycol(14) isostearyl ether, polyethylene glycol(15) isostearyl ether, polyethylene glycol(16) isostearyl ether, polyethylene glycol(17) isostearyl ether, polyethylene glycol(18) isostearyl ether, polyethylene glycol(19) isostearyl ether, polyethylene glycol(20) isostearyl ether, polyethylene glycol(13) cetyl ether, polyethylene glycol(14) cetyl ether, polyethylene glycol(15) cetyl ether, polyethylene glycol(16) cetyl ether, polyethylene glycol(17) cetyl ether, polyethylene glycol(18) cetyl ether, polyethylene glycol(19) cetyl ether, polyethylene glycol(20) cetyl ether, polyethylene glycol(13) isocetyl ether, polyethylene glycol(14) isocetyl ether, polyethylene glycol(15) isocetyl ether, polyethylene glycol(16) isocetyl ether, polyethylene glycol(17) isocetyl ether, polyethylene glycol(18) isocetyl ether, polyethylene glycol(19) isocetyl ether, polyethylene glycol(20) isocetyl ether, polyethylene glycol(12) oleyl ether, polyethylene glycol(13) oleyl ether, polyethylene glycol(14) oleyl ether, polyethylene glycol(15) oleyl ether, polyethylene glycol(12) lauryl ether, polyethylene glycol(12) isolauryl ether, polyethylene glycol(13) cetylstearyl ether, polyethylene glycol(14) cetylstearyl ether, polyethylene glycol(15) cetylstearyl ether, polyethylene glycol(16) cetylstearyl ether, polyethylene glycol(17) cetylstearyl ether, polyethylene glycol(18) cetylstearyl ether, polyethylene glycol(19) cetylstearyl ether.

Fatty acid ethoxylates selected from the group consisting of ethoxylated stearates, isostearates and oleates, in particular polyethylene glycol(20) stearate, polyethylene glycol(21) stearate, polyethylene glycol(22) stearate, polyethylene glycol(23) stearate, polyethylene glycol(24) stearate, polyethylene glycol(25) stearate, polyethylene glycol(12) isostearate, polyethylene glycol(13) isostearate, polyethylene glycol(14) isostearate, polyethylene glycol(15) isostearate, polyethylene glycol(16) isostearate, polyethylene glycol(17) isostearate, polyethylene glycol(18) isostearate, polyethylene glycol(19) isostearate, polyethylene glycol(20) isostearate, polyethylene glycol(21) isostearate, polyethylene glycol(22) isostearate, polyethylene glycol(23) isostearate, polyethylene glycol(24) isostearate, polyethylene glycol(25) isostearate, polyethylene glycol(12) oleate, polyethylene glycol(13) oleate, polyethylene glycol(14) oleate, polyethylene glycol(15) oleate, polyethylene glycol(16) oleate, polyethylene glycol(17) oleate, polyethylene glycol(18) oleate, polyethylene glycol(19) oleate, polyethylene glycol(20)oleate, are likewise preferred.

Sodium laureth-11 carboxylate can advantageously be used as ethoxylated alkylether carboxylic acid or salts thereof.

Ethoxylated triglycerides which can be used advantageously are polyethylene glycol(60) evening primrose glycerides.

It is furthermore advantageous to select the polyethylene glycol glycerol fatty acid esters from the group consisting of polyethylene glycol(20) glyceryl laurate, polyethylene glycol(6) glyceryl caprate/caprinate, polyethylene glycol(20) glyceryl oleate, polyethylene glycol(20) glyceryl isostearate and polyethylene glycol(18) glyceryl oleate/cocoate.

From among the ethoxylated sorbitan esters, polyethylene glycol(20) sorbitan monolaurate, polyethylene glycol(20) sorbitan monostearate, polyethylene glycol(20) sorbitan monoisostearate, polyethylene glycol(20) sorbitan monopalmitate, polyethylene glycol(20) sorbitan monooleate are particularly suitable.

Particularly advantageous coemulsifiers are glyceryl monostearate, glyceryl monooleate, diglyceryl monostearate, glyceryl isostearate, polyglyceryl-3 oleate, polyglyceryl-3 diisostearate, polyglyceryl-4 isostearate, polyglyceryl-2 dipolyhydroxystearate, polyglyceryl-4 dipolyhydroxystearate, PEG-30 dipolyhydroxystearate, diisostearoyl polyglyceryl-3 diisostearate, glycol distearate and polyglyceryl-3 dipolyhydroxystearate, sorbitan monoisostearate, sorbitan stearate, sorbitan oleate, sucrose distearate, lecithin, PEG-7-hydrogenated castor oil, cetyl alcohol, stearyl alcohol, behenyl alcohol, isobehenyl alcohol and polyethylene glycol(2) stearyl ether (steareth-2), alkylmethicone copolyols and alkyldimethicone copolyols, in particular cetyldimethicone copolyol (ABIL® EM 90), laurylmethicone copolyol or amodimethicone glycerocarbamate (SilCare® Silicone WSI, Clariant).

If the cosmetic, dermatological or pharmaceutical compositions according to the invention comprise one or more substances selected from the group consisting of emulsifiers, coemulsifiers and solubilizers, this one substance or these substances is/are preferably present in the compositions according to the invention in an amount of from 0.1 to 20.0% by weight, particularly preferably in an amount of from 0.5 to 10.0% by weight and especially preferably in an amount of from 1.0 to 5.0% by weight, based on the total weight of the respective composition according to the invention.

Suitable for use as electrolyte are inorganic salts, preferably ammonium salts or metal salts, particularly preferably of halides, for example CaCl₂, MgCl₂, LiCl, KCl and NaCl, carbonates, bicarbonates, phosphates, sulfates, nitrates, especially preferably sodium chloride, and/or organic salts, preferably ammonium salts or metal salts, particularly preferably of glycolic acid, lactic acid, citric acid, tartaric acid, mandelic acid, salicylic acid, ascorbic acid, pyruvic acid, fumaric acid, retinoic acid, sulfonic acids, benzoic acid, kojic acid, fruit acid, malic acid, gluconic acid or galacturonic acid.

These also include aluminum salts, preferably aluminum chlorohydrate or aluminum-zirkonium complex salts.

Accordingly, in a preferred embodiment of the invention, the cosmetic, dermatological or pharmaceutical compositions according to the invention comprise one or more substances selected from the group consisting of inorganic and organic salts.

As electrolytes, the cosmetic, dermatological or pharmaceutical compositions according to the invention may also comprise mixtures of different salts.

If the cosmetic, dermatological or pharmaceutical compositions according to the invention comprise one or more electrolytes, these are preferably present in the compositions according to the invention in an amount of from 0.01 to 20.0% by weight, particularly preferably in an amount of from 0.1 to 10.0% by weight and especially preferably in an amount of from 0.5 to 5.0% by weight, based on the total weight of the respective composition according to the invention.

In a further preferred embodiment of the invention, the cosmetic, dermatological or pharmaceutical compositions according to the invention comprise one or more hydroxy acids, particularly preferably one or more substances selected from the group consisting of alpha- and beta-hydroxy acids.

As hydroxy acids, the cosmetic, dermatological or pharmaceutical compositions according to the invention may may preferably comprise lactic acid, glycolic acid, salicylic acid and alkylated salicylic acids or citric acid. The cosmetic, dermatological or pharmaceutical compositions according to the invention may additionally comprise further acidic components. Suitable active compounds are tartaric acid, mandelic acid, caffeic acid, pyruvic acid, oligooxamono- and -dicarboxylic acids, fumaric acid, retinoic acid, sulfonic acids, benzoic acid, kojic acid, fruit acid, malic acid, gluconic acid, pyruvic acid, galacturonic acid, ribonic acid, and all their derivatives, polyglycoldioic acids in free or partially neutralized form, vitamin C (ascorbic acid), vitamin C derivatives, dihydroxyacetone or skin-whitening actives such as arbutin or glycyrrhetic acid and salts thereof. If the cosmetic, dermatological or pharmaceutical compositions according to the invention comprise one or more of these substances just mentioned, this one substance or these substances is/are preferably present in the compositions according to the invention in an amount of from 0.1 to 20.0% by weight, particularly preferably in an amount of from 0.2 to 10.0% by weight and especially preferably in an amount of from 0.5 to 5.0% by weight, based on the total weight of the respective composition according to the invention.

In a further preferred embodiment of the invention, the cosmetic, dermatological or pharmaceutical compositions according to the invention therefore comprise one or more substances selected from the group consisting of vitamin C and vitamin C derivatives, where the vitamin C derivatives are preferably selected from the group consisting of sodium ascorbylphosphate, magnesium ascorbylphoshate and magnesium ascorbylglucoside.

In a further preferred embodiment of the invention, the cosmetic, dermatological or pharmaceutical compositions according to the invention comprise one or more substances selected from the group consisting of benzoic acid, sorbic acid, salicylic acid, lactic acid and paramethoxybenzoic acid. The organic acids mentioned above may serve as further preservatives.

Stabilizers which can be used in the cosmetic, dermatological or pharmaceutical compositions according to the invention are metal salts of fatty acids, such as, for example, magnesium stearate, aluminum stearate and/or zinc stearate. If the cosmetic, dermatological or pharmaceutical compositions according to the invention comprise one or more of these substances just mentioned, this one substance or these substances is/are preferably present in the compositions according to the invention in an amount of from 0.1 to 10.0% by weight, particularly preferably in an amount of from 0.5 to 8.0% by weight and especially preferably in an amount of from 1.0 to 5.0% by weight, based on the total weight of the respective composition according to the invention.

Suitable cationic polymers are the substances known under the INCI name “polyquaternium”, in particular polyquaternium-31, polyquaternium-16, polyquaternium-24, polyquaternium-7, polyquaternium-22, polyquaternium-39, polyquaternium-28, polyquaternium-2, polyquaternium-10, polyquaternium-11, and polyquaternium 37&mineral oil & PPG trideceth (Salcare SC95), PVP-dimethylaminoethyl methacrylate copolymer, guar hydroxypropyltriammonium chlorides, and calcium alginate and ammonium alginate. Furthermore, use may be made of cationic cellulose derivatives; cationic starch; copolymers of diallylammonium salts and acrylamides; quaternized vinylpyrrolidone/vinylimidazole polymers; condensation products of polyglycols and amines; quaternized collagen polypeptides; quaternized wheat polypeptides; polyethyleneimines; cationic silicone polymers, such as, for example, amidomethicones; copolymers of adipic acid and dimethylaminohydroxypropyldiethylenetriamine; polyaminopolyamide and cationic chitin derivatives, such as, for example, chitosan.

If the cosmetic, dermatological or pharmaceutical compositions according to the invention comprise one or more of the cationic polymers mentioned above, these are preferably present in the compositions according to the invention in an amount of from 0.1 to 5.0% by weight, particularly preferably in an amount of from 0.2 to 3.0% by weight and especially preferably in an amount of from 0.5 to 2.0% by weight, based on the total weight of the respective composition according to the invention.

Furthermore, the cosmetic, dermatological or pharmaceutical compositions according to the invention may comprise film formers which, depending on the intended use, are selected from salts of phenylbenzimidazolesulfonic acid, water-soluble polyurethanes, for example C₁₀-polycarbamyl polyglyceryl ester, polyvinyl alcohol, polyvinylpyrrolidone copolymers, for example vinylpyrrolidone/vinyl acetate copolymer, or PVP/eicosene copolymers, maleinated polypropylene polymers, water-soluble acrylic acid polymers/copolymers and esters or salts thereof, for example partial ester copolymers of acrylic/methacrylic acid, water-soluble cellulose, for example hydroxymethylcellulose, hydroxyethylcellulose, hydroxypropylcellulose, water-soluble quaterniums, polyquaterniums, carboxyvinyl polymers, such as carbomers and salts thereof, polysaccharides, for example polydextrose and glucan, vinyl acetate/crotonate, for example available under the trade name Aristoflex® A 60 (Clariant).

If the cosmetic, dermatological or pharmaceutical compositions according to the invention comprise one or more film formers, these are preferably present in the compositions according to the invention in an amount of from 0.1 to 10.0% by weight, particularly preferably in an amount of from 0.2 to 5.0% by weight and especially preferably in an amount of from 0.5 to 3.0% by weight, based on the total weight of the respective composition according to the invention.

The desired viscosity of the cosmetic, dermatological or pharmaceutical compositions can be established by adding thickeners and gelling agents. Suitable are preferably cellulose ethers and other cellulose derivatives (e.g. carboxymethylcellulose, hydroxyethylcellulose), gelatin, starch and starch derivatives, sodium alginates, fatty acid polyethylene glycol esters, agar, carrageenan, tragacanth or dextrin derivatives, in particular dextrin esters. Furthermore suitable are metal salts of fatty acids, preferably having 12 to 22 carbon atoms, for example sodium stearate, sodium palmitate, sodium laurate, sodium arachidates, sodium behenate, potassium stearate, potassium palmitate, sodium myristate, aluminum monostearate, hydroxy fatty acids, for example 12-hydroxystearic acid, 16-hydroxyhexadecanoyl acid; fatty acid amides; fatty acid alkanolamides; dibenzalsorbitol and alcohol-soluble polyamides and polyacrylamides or mixtures of such. Use may furthermore be made of crosslinked and uncrosslinked polyacrylates such as carbomers, sodium polyacrylates or polymers containing sulfonic acid, such as ammonium acryloyldimethyltaurate/VP copolymer or sodium acryloyldimethyltaurate/VP copolymer.

If the cosmetic, dermatological or pharmaceutical compositions according to the invention comprise one or more substances selected from the group consisting of thickeners and gelling agents, this one substance or these substances is/are preferably present in the compositions according to the invention in an amount of from 0.01 to 20.0% by weight, particularly preferably in an amount of from 0.1 to 10.0% by weight, especially preferably in an amount of from 0.2 to 3.0% by weight and most preferably in an amount of from 0.4 to 2.0% by weight, based on the total weight of the respective composition according to the invention.

Preferred for use as superfattening agents or refattening agents are lanolin and lecithin, non-ethoxylated and polyethoxylated or acylated lanolin and lecithin derivatives, polyol fatty acid esters such as glyceryl oleate, mono-, di- and triglycerides and/or fatty acid alkanolamides, the latter simultaneously serving as foam stabilizers. If the cosmetic, dermatological or pharmaceutical compositions according to the invention comprise one or more of the substances just mentioned, this one substance or these substances is/are preferably present in the compositions according to the invention in an amount of from 0.01 to 10.0% by weight, particularly preferably in an amount of from 0.1 to 5.0% by weight and especially preferably in an amount of from 0.5 to 3.0% by weight, based on the total weight of the respective composition according to the invention.

In a further preferred embodiment of the invention, the cosmetic, dermatological or pharmaceutical compositions according to the invention comprise one or more antimicrobially active compounds and are preferably present in the form of disinfectant compositions and particularly preferably in the form of disinfectant gels.

Antimicrobially active compounds employed may be cetyltrimethylammonium chloride, cetylpyridinium chloride, benzethonium chloride, diisobutylethoxyethyldimethylbenzylammonium chloride, sodium N-laurylsarcosinate, sodium N-palmethylsarcosinate, lauroylsarcosine, N-myristoylglycine, potassium N-laurylsarcosine, trimethylammonium chloride, sodium aluminum chlorohydroxylactate, triethyl citrate, tricetylmethylammonium chloride, 2,4,4′-trichloro-2′-hydroxydiphenyl ether (triclosan), phenoxyethanol, 1,5-pentanediol, 1,6-hexanediol, 3,4,4′-trichlorocarbanilide (triclocarban), diaminoalkylamide, for example L-lysine hexadecylamide, citrate heavy metal salts, salicylates, piroctoses, in particular zinc salts, pyrithiones and heavy metal salts thereof, in particular zinc pyrithione, zinc phenol sulfate, farnesol, ketoconazole, oxiconazole, bifonazole, butoconazole, cloconazole, clotrimazole, econazole, enilconazole, fenticonazole, isoconazole, miconazole, sulconazole, tioconazole, fluconazole, itraconazole, terconazole, naftifine and terbinafine, selenium disulfide and Octopirox®, methylchloroisothiazolinone, methylisothiazolinone, methyldibromoglutaronitrile, AgCl, chloroxylenol, Na salt of diethylhexyl sulfosuccinate, sodium benzoate, and phenoxyethanol, benzyl alcohol, phenoxyisopropanol, parabens, preferably butyl, ethyl, methyl and propyl paraben, and Na salts thereof, pentanediol, 1,2-octanediol, ethylhexylglycerol, benzyl alcohol, sorbic acid, benzoic acid, lactic acid, imidazolidinylurea, diazolidinylurea, dimethyloldimethylhydantoin (DMDMH), Na salt of hydroxymethylglycinate, hydroxyethylglycine of sorbic acid and combinations of these active substances.

If the cosmetic, dermatological or pharmaceutical compositions according to the invention comprise one or more antimicrobially active compounds, these are preferably present in the compositions according to the invention in an amount of from 0.001 to 5.0% by weight, particularly preferably in an amount of from 0.01 to 3.0% by weight and especially preferably in an amount of from 0.1 to 2.0% by weight, based on the total weight of the respective composition according to the invention.

The cosmetic, dermatological or pharmaceutical compositions according to the invention may furthermore comprise biogenic active compounds selected from plant extracts, such as, for example, aloe vera, and also local anesthetics, antibiotics, antiphlogistics, antiallergics, corticosteroids, sebostatics, Bisabolol®, allantoin, Phytantriol, proteins, vitamins selected from niacin, biotin, vitamin B2, vitamin B3, vitamin B6, vitamin B3 derivatives (salts, acids, esters, amides, alcohols), vitamin C and vitamin C derivatives (salts, acids, esters, amides, alcohols), preferably as sodium salt of the monophosphoric acid ester of ascorbic acid or as magnesium salt of the phosphoric acid ester of ascorbic acid, tocopherol and tocopherol acetate, and also vitamin E and/or derivatives thereof.

If the cosmetic, dermatological or pharmaceutical compositions according to the invention comprise one or more biogenic active compounds, these are preferably present in the compositions according to the invention in an amount of from 0.001 to 5.0% by weight, particularly preferably in an amount of from 0.01 to 3.0% by weight and especially preferably in an amount of from 0.1 to 2.0% by weight, based on the total weight of the respective composition according to the invention.

The cosmetic, dermatological or pharmaceutical compositions according to the invention may comprise astringents, preferably magnesium oxide, aluminum oxide, titanium dioxide, zirconium dioxide and zinc oxide, oxide hydrates, preferably aluminum oxide hydrate (boehmite) and hydroxides, preferably of calcium, magnesium, aluminum, titanium, zirconium or zinc, and also aluminum chlorohydrates. If the cosmetic, dermatological or pharmaceutical compositions according to the invention comprise one or more astringents, these are preferably present in the compositions according to the invention in an amount of from 0.001 to 50.0% by weight, particularly preferably in an amount of from 0.01 to 10.0% by weight and especially preferably in an amount of from 0.1 to 10.0% by weight, based on the total weight of the respective composition according to the invention.

Preferred deodorizing substances are allantoin and bisabolol. If the cosmetic, dermatological or pharmaceutical compositions according to the invention comprise one or more deodorizing substances, these are preferably present in the compositions according to the invention in an amount of from 0.0001 to 10.0% by weight, based on the total weight of the respective composition according to the invention.

The cosmetic, dermatological or pharmaceutical compositions according to the invention may comprise one or more antioxidants, preferably selected from the group consisting of amino acids (e.g. glycine, histidine, tyrosine, tryptophan) and derivatives thereof, imidazoles (e.g. urocanic acid) and derivatives thereof, peptides such as D, L-carnosine, D-carnosine, L-carnosine and derivatives thereof (e.g. anserine), carotenoids, carotenes (e.g. α-carotene, β-carotene, lycopene) and derivatives thereof, chlorogenic acid and derivatives thereof, lipoic acid and derivatives thereof (e.g. dihydrolipoic acid), aurothioglucose, propylthiouracil and other thiols (e.g, thioredoxin, glutathione, cysteine, cystine, cystamine and the glycosyl, N-acetyl, methyl, ethyl, propyl, amyl, butyl and lauryl, palmitoyl, oleyl, γ-linoleyl, cholesteryl and glyceryl esters thereof), and also salts thereof, dilauryl thiodipropionate, distearyl thiodipropionate, thiodipropionic acid and derivatives thereof (e.g. esters, ethers, peptides, lipids, nucleotides, nucleosides and salts), and also sulfoximine compounds (e.g. buthionine sulfoximines, homocysteine sulfoximine, buthionine sulfones, penta-, hexa-, heptathionine sulfoximine) in very low tolerated doses, also (metal) chelating agents (e.g. α-hydroxy fatty acids, palmitic acid, phytic acid, lactoferrin), α-hydroxy acids (e.g. citric acid, lactic acid, malic acid), humic acid, bile acid, bile extracts, bilirubin, biliverdin, EDTA, EGTA and derivatives thereof, unsaturated fatty acids and derivatives thereof (e.g. γ-linolenic acid, linoleic acid, oleic acid), folic acid and derivatives thereof, ubiquinone and ubiquinol and derivatives thereof, vitamin C and derivatives (e.g. ascorbyl palmitate, Mg ascorbyl phosphate, ascorbyl acetate), tocopherols and derivatives (e.g. vitamin E acetate), vitamin A and derivatives (vitamin A palmitate), and coniferyl benzoate of benzoin resin, rutinic acid and derivatives thereof, α-glycosylrutin, ferulic acid, furfurylideneglucitol, carnosine, butylhydroxytoluene, butylhydroxyanisole, nordihydroguaiacic acid, nordihydroguaiaretic acid, trihydroxybutyrophenone, uric acid and derivatives thereof, mannose and derivatives thereof, zinc and derivatives thereof (e.g. ZnO, ZnSO₄), selenium and derivatives thereof (e.g. selenomethionine), stilbenes and derivatives thereof (e.g. stilbene oxide, trans-stilbene oxide), superoxide dismutase and the derivatives suitable according to the invention (salts, esters, ethers, sugars, nucleotides, nucleosides, peptides and lipids) of these specified substances.

The antioxidants can protect the skin and the hair against oxidative stress. Preferred antioxidants here are vitamin E and derivatives thereof, and vitamin A and derivatives thereof.

If the cosmetic, dermatological or pharmaceutical compositions according to the invention comprise one or more antioxidants, these are preferably present in the compositions according to the invention in an amount of from 0.001 to 30.0% by weight, particularly preferably in an amount of from 0.05 to 20.0% by weight and especially preferably in an amount of from 1.0 to 10.0% by weight, based on the total weight of the respective composition according to the invention.

Furthermore, humectants selected from the group consisting of the sodium salt of 2-pyrrolidone-5-carboxylate (NaPCA), guanidine; glycolic acid and salts thereof, lactic acid and salts thereof, glucosamines and salts thereof, lactamide monoethanolamine, acetamide monoethanolamine, urea, hydroxyethyl urea, hydroxy acids, panthenol and derivatives thereof, for example D-panthenol (R-2,4-dihydroxy-N-(3-hydroxypropyl)-3,3-dimethylbutamide), D, L-panthenol, calcium pantothenate, panthetine, pantotheine, panthenyl ethyl ether, isopropyl palmitate and/or glycerol may be employed. If the compositions according to the invention comprise one or more humectants, these are preferably present in the compositions according to the invention in an amount of from 0.1 to 15.0% by weight and particularly preferably in an amount of from 0.5 to 5.0% by weight, based on the total weight of the respective composition according to the invention.

The cosmetic, dermatological or pharmaceutical compositions according to the invention may additionally comprise organic solvents. In principle, suitable organic solvents are all mono- or polyhydric alcohols. Preference is given to alcohols having 1 to 4 carbon atoms, such as ethanol, propanol, isopropanol, n-butanol, isobutanol, tert-butanol, glycerol and mixtures of said alcohols. Further preferred alcohols are polyethylene glycols with a relative molecular mass below 2000. In particular, a use of polyethylene glycol with a relative molecular mass between 200 and 600 and in amounts up to 45.0% by weight and of polyethylene glycol with a relative molecular mass between 400 and 600 in amounts of from 5.0 to 25.0% by weight is preferred. Further suitable solvents are, for example, triacetin (glycerol triacetate) and 1-methoxy-2-propanol.

The cosmetic, dermatological or pharmaceutical compositions according to the invention may comprise one or more substances selected from colorants, e.g. dyes and/or pigments. The dyes and/or pigments present in the cosmetic, dermatological or pharmaceutical compositions according to the invention, both organic and inorganic dyes and pigments, are selected from the corresponding positive list of the Cosmetics Regulations or the EU list of cosmetic colorants.

| Chemical or other name | CIN | Color |
|---|---|---|
| Pigment Green | 10006 | green |
| Acid Green 1 | 10020 | green |
| 2,4-dinitrohydroxynaphthalene-7-sulfonic acid | 10316 | yellow |
| Pigment Yellow 1 | 11680 | yellow |
| Pigment Yellow 3 | 11710 | yellow |
| Pigment Orange 1 | 11725 | orange |
| 2,4-dihydroxyazobenzene | 11920 | orange |
| Solvent Red 3 | 12010 | red |
| 1-(2′-chloro-4′-nitro-1′-phenylazo)-2- | 12085 | red |
| hydroxynaphthalene | | |
| Pigment Red 3 | 12120 | red |
| Ceres Red; Sudan Red; Fat Red G | 12150 | red |
| Pigment Red 112 | 12370 | red |
| Pigment Red 7 | 12420 | red |
| Pigment Brown 1 | 12480 | brown |
| 4-(2′-methoxy-5′-sulfonic acid diethylamide-1′- | 12490 | red |
| phenylazo)-3-hydroxy-5″-chloro-2″,4″-dimethoxy-2- | | |
| naphthoic anilide | | |
| Disperse Yellow 16 | 12700 | yellow |
| 1-(4-sulfo-1-phenylazo)-4-aminobenzenesulfonic acid | 13015 | yellow |
| 2,4-dihydroxyazobenzene-4′-sulfonic acid | 14270 | orange |
| 2-(2,4-dimethylphenylazo-5-sulfonic acid)-1- | 14700 | red |
| hydroxynaphthalene-4-sulfonic acid | | |
| 2-(4-sulfo-1-naphthylazo)-1-naphthol-4-sulfonic acid | 14720 | red |
| 2-(6-sulfo-2,4-xylylazo)-1-naphthol-5-sulfonic acid | 14815 | red |
| 1-(4′-sulfophenylazo)-2-hydroxynaphthalene | 15510 | orange |
| 1-(2-sulfonic acid-4-chloro-5-carboxylic acid-1- | 15525 | red |
| phenylazo)-2-hydroxynaphthalene | | |
| 1-(3-methylphenylazo-4-sulfonic acid)-2- | 15580 | red |
| hydroxynaphthalene | | |
| 1-(4′,(8′)-sulfonic acid naphthylazo)-2- | 15620 | red |
| hydroxynaphthalene | | |
| 2-hydroxy-1,2′-azonaphthalene-1′-sulfonic acid | 15630 | red |
| 3-hydroxy-4-phenylazo-2-naphthylcarboxylic acid | 15800 | red |
| 1-(2-sulfo-4-methyl-1-phenylazo)-2- | 15850 | red |
| naphthylcarboxylic acid | | |
| 1-(2-sulfo-4-methyl-5-chloro-1-phenylazo)-2- | 15865 | red |
| hydroxynaphthalene-3-carboxylic acid | | |
| 1-(2-sulfo-1-naphthylazo)-2-hydroxynaphthalene-3- | 15880 | red |
| carboxylic acid | | |
| 1-(3-sulfo-1-phenylazo)-2-naphthol-6-sulfonic acid | 15980 | orange |
| 1-(4-sulfo-1-phenylazo)-2-naphthol-6-sulfonic acid | 15985 | yellow |
| Allura Red | 16035 | red |
| 1-(4-sulfo-1-naphthylazo)-2-naphthol-3,6-disulfonic | 16185 | red |
| acid | | |
| Acid Orange 10 | 16230 | orange |
| 1-(4-sulfo-1-naphthylazo)-2-naphthol-6,8-disulfonic | 16255 | red |
| acid | | |
| 1-(4-sulfo-1-naphthylazo)-2-naphthol-3,6,8-trisulfonic | 16290 | red |
| acid | | |
| 8-amino-2-phenylazo-1-naphthol-3,6-disulfonic acid | 17200 | red |
| Acid Red 1 | 18050 | red |
| Acid Red 155 | 18130 | red |
| Acid Yellow 121 | 18690 | yellow |
| Acid Red 180 | 18736 | red |
| Acid Yellow 11 | 18820 | yellow |
| Acid Yellow 17 | 18965 | yellow |
| 4-(4-sulfo-1-phenylazo)-1-(4-sulfophenyl)-5- | 19140 | yellow |
| hydroxyphrazolone-3-carboxylic acid | | |
| Pigment Yellow 16 | 20040 | yellow |
| 2,6-(4′-sulfo-2″,4″-dimethyl)-bis-phenylazo)1,3- | 20170 | orange |
| dihydroxybenzene | | |
| Acid Black 1 | 20470 | black |
| Pigment Yellow 13 | 21100 | yellow |
| Pigment Yellow 83 | 21108 | yellow |
| Solvent Yellow | 21230 | yellow |
| Acid Red 163 | 24790 | red |
| Acid Red 73 | 27290 | red |
| 2-[4′-(4″-sulfo-1″-phenylazo)-7′-sulfo-1′-naphthylazo]- | 27755 | black |
| 1-hydroxy-7-aminonaphthalene-3,6-disulfonic acid | | |
| 4′-[(4″-sulfo-1″-phenylazo)-7′-sulfo-1′-naphthylazo]-1- | 28440 | black |
| hydroxy-8-acetylaminonaphthalene-3,5-disulfonic acid | | |
| Direct Orange 34, 39, 44, 46, 60 | 40215 | orange |
| Food Yellow | 40800 | orange |
| trans-β-apo-8′-carotinaldehyde (C₃₀) | 40820 | orange |
| trans-apo-8′-carotinic acid (C₃₀)-ethyl ester | 40825 | orange |
| canthaxanthin | 40850 | orange |
| Acid Blue 1 | 42045 | blue |
| 2,4-disulfo-5-hydroxy-4′-4″- | 42051 | blue |
| bis(diethylamino)triphenylcarbinol | | |
| 4-[(4-N-ethyl-p-sulfobenzylamino)phenyl(4-hydroxy- | 42053 | green |
| 2-sulfophenyl)(methylene)-1-(N-ethyl-N-p- | | |
| sulfobenzyl)-2,5-cyclohexadienimine] | | |
| Acid Blue 7 | 42080 | blue |
| (N-ethyl-p-sulfobenzylaminophenyl-(2- | 42090 | blue |
| sulfophenyl)methylene-(N-ethyl-N-p- | | |
| sulfobenzyl)cyclohexadienimine | | |
| Acid Green 9 | 42100 | green |
| diethyldisulfobenzyl-di-4-amino-2-chlorodi-2- | 42170 | green |
| methylfuchsonimmonium | | |
| Basic Violet 14 | 42510 | violet |
| Basic Violet 2 | 42520 | violet |
| 2′-methyl-4′-(N-ethyl-N-m-sulfobenzyl)amino-4″-(N- | 42735 | blue |
| diethyl)amino-2-methyl-N-ethyl-N-m- | | |
| sulfobenzylfuchsonimmonium | | |
| 4′-(N-dimethyl)amino-4″-(N-phenyl)aminonaphtho-N- | 44045 | blue |
| dimethylfuchsonimmonium | | |
| 2-hydroxy-3,6-disulfo-4,4′- | 44090 | green |
| bisdimethylaminonaphthofuchsonimmonium | | |
| Acid Red | 45100 | red |
| 3-(2′-methylphenylamino)-6-(2′-methyl-4′- | 45190 | violet |
| sulfophenylamino)-9-(2″-carboxyphenyl)xanthenium | | |
| salt | | |
| Acid Red 50 | 45220 | red |
| phenyl-2-oxyfluorone-2-carboxylic acid | 45350 | yellow |
| 4,5-dibromofluorescein | 45370 | orange |
| 2,4,5,7-tetrabromofluorescein | 45380 | red |
| Solvent Dye | 45396 | orange |
| Acid Red 98 | 45405 | red |
| 3′,4′,5′,6′-tetrachloro-2,4,5,7-tetrabromofluorescein | 45410 | red |
| 4,5-diiodofluorescein | 45425 | red |
| 2,4,5,7-tetraiodofluorescein | 45430 | red |
| quinophthalone | 47000 | yellow |
| quinophthalonedisulfonic acid | 47005 | yellow |
| Acid Violet 50 | 50325 | violet |
| Acid Black 2 | 50420 | black |
| Pigment Violet 23 | 51319 | violet |
| 1,2-dioxyanthraquinone, calcium-aluminum complex | 58000 | red |
| 3-oxypyrene-5,8,10-sulfonic acid | 59040 | green |
| 1-hydroxy-4-N-phenylaminoanthraquinone | 60724 | violet |
| 1-hydroxy-4-(4′-methylphenylamino)anthraquinone | 60725 | violet |
| Acid Violet 23 | 60730 | violet |
| 1,4-di(4′-methylphenylamino)anthraquinone | 61565 | green |
| 1,4-bis-(o-sulfo-p-toluidine)anthraquinone | 61570 | green |
| Acid Blue 80 | 61585 | blue |
| Acid Blue 62 | 62045 | blue |
| N,N′-dihydro-1,2,1′,2′-anthraquinonazine | 69800 | blue |
| Vat Blue 6; Pigment Blue 64 | 69825 | blue |
| Vat Orange 7 | 71105 | orange |
| indigo | 73000 | blue |
| indigodisulfonic acid | 73015 | blue |
| 4,4′-dimethyl-6,6′-dichlorothioindigo | 73360 | red |
| 5,5′-dichloro-7,7′-dimethylthioindigo | 73385 | violet |
| Quinacridone Violet 19 | 73900 | violet |
| Pigment Red 122 | 73915 | red |
| Pigment Blue 16 | 74100 | blue |
| phthalocyanine | 74160 | blue |
| Direct Blue 86 | 74180 | blue |
| chlorinated phthalocyanines | 74260 | green |
| Natural Yellow 6,19; Natural Red 1 | 75100 | yellow |
| bixin, norbixin | 75120 | orange |
| lycopene | 75125 | yellow |
| trans-alpha, beta- or gamma-carotene | 75130 | orange |
| keto- and/or hydroxyl derivatives of carotene | 75135 | yellow |
| guanine or pearlizing agents | 75170 | white |
| 1,7-bis-(4-hydroxy-3-methoxyphenyl)-1,6-heptadiene- | 75300 | yellow |
| 3,5-dione | | |
| complex salt (Na,Al,Ca) of carminic acid | 75470 | red |
| chlorophyll a and b; copper compounds of chlorophylls | 75810 | green |
| and chlorophyllins | | |
| aluminum | 77000 | white |
| alumina hydrate | 77002 | white |
| water-containing aluminum silicates | 77004 | white |
| ultramarine | 77007 | blue |
| Pigment Red 101 and 102 | 77015 | red |
| barium sulfate | 77120 | white |
| bismuth oxychloride and mixtures thereof with mica | 77163 | white |
| calcium carbonate | 77220 | white |
| calcium sulfate | 77231 | white |
| carbon | 77266 | black |
| Pigment Black 9 | 77267 | black |
| Carbo medicinalis vegetabilis | 77268:1 | black |
| chromium oxide | 77288 | green |
| chromium oxide, water-containing | 77289 | green |
| Pigment Blue 28, Pigment Green 14 | 77346 | green |
| Pigment Metal 2 | 77400 | brown |
| gold | 77480 | brown |
| iron oxides and hydroxides | 77489 | orange |
| iron oxides and hydroxides | 77491 | red |
| iron oxide hydrate | 77492 | yellow |
| iron oxide | 77499 | black |
| mixtures of iron(II) and iron(III) hexacyanoferrate | 77510 | blue |
| Pigment White 18 | 77713 | white |
| manganese ammonium diphosphate | 77742 | violet |
| manganese phosphate; Mn₃(PO₄)₂*7H₂O | 77745 | red |
| silver | 77820 | white |
| titanium dioxide and mixtures thereof with mica | 77891 | white |
| zinc oxide | 77947 | white |
| 6,7-dimethyl-9-(1′-D-ribityl)isoalloxazine, lactoflavin | | yellow |
| caramel | | brown |
| capsanthin, capsorubin | | orange |
| betanin | | red |
| benzopyrilium salts, anthocyanines | | red |
| aluminum stearate, zinc stearate, magnesium stearate | | white |
| and calcium stearate | | |
| Bromothymol Blue | | blue |
| Bromocresol Green | | green |
| Acid Red 195 | | red |

Oil-soluble natural dyes, such as, for example, paprika extracts, β-carotene and cochineal are furthermore advantageous.

Also advantageously used are pearlescent pigments, e.g. pearl essence (guanine/hypoxanthine mixed crystals from fish scales) and mother of pearl (ground seashells), monocrystalline pearlescent pigments such as, for example, bismuth oxychloride (BiOCl), layer substrate pigments, e.g. mica/metal oxide, silver-white pearlescent pigments from TiO₂, interference pigments (TiO₂, variable layer thickness), color luster pigments (Fe₂O₃) and combination pigments (TiO₂/Fe₂O₃, TiO₂/Cr₂O₃, TiO₂/Prussian blue, TiO₂/carmine).

Effect pigments within the context of the present invention are understood as meaning pigments which by virtue of their refraction properties produce special optical effects. Effect pigments impart to the treated surface (skin, hair, mucous membrane) luster or glitter effects or can visually conceal unevenness of the skin and skin wrinkles by means of diffuse light scattering. As a particular embodiment of the effect pigments, interference pigments are preferred. Particularly suitable effect pigments are, for example, mica particles which are coated with at least one metal oxide. Besides mica, a sheet silicate, silica gel and other SiO₂ modifications are also suitable as carriers. A metal oxide frequently used for coating is, for example, titanium oxide, to which, if desired, iron oxide can be admixed. By means of the size and shape (e.g. spherical, ellipsoidal, flat, even, uneven) of the pigment particles and by means of the thickness of the oxide coating, the reflection properties can be influenced. Other metal oxides, e.g. bismuth oxychloride (BiOCl), and the oxides of, for example, titanium, in particular the TiO₂ modifications anatase and rutile, aluminum, tantalum, niobium, zirconium and hafnium can also be used. Effect pigments can also be prepared using magnesium fluoride (MgF₂) and calcium fluoride (fluorspar, CaF₂).

The effects can be controlled both by means of the particle size and by means of the particle size distribution of the pigment ensemble. Suitable particle size distributions extend, for example, from 2-50 μm, 5-25 μm, 5-40 μm, 5-60 μm, 5-95 μm, 5-100 μm, 10-60 μm, 10-100 μm, 10-125 μm, 20-100 μm, 20-150 μm, and <15 μm. A wider particle size distribution, for example of 20-150 μm, produces glittering effects, whereas a narrower particle size distribution of <15 μm provides for a uniform silky appearance.

If the cosmetic, dermatological or pharmaceutical compositions according to the invention comprise one or more effect pigments, these are preferably present in the compositions according to the invention in an amount of from 0.1 to 20.0% by weight, particularly preferably in an amount of from 0.5 to 10.0% by weight and especially preferably in an amount of from 1.0 to 5.0% by weight, based on the total weight of the respective composition according to the invention.

Preferably suitable as pearlizing component are fatty acid monoalkanolamides, fatty acid dialkanolamides, monoesters or diesters of alkylene glycols, in particular ethylene glycol and/or propylene glycol or oligomers thereof, with higher fatty acids, such as, for example, palmitic acid, stearic acid and behenic acid, monoesters or polyesters of glycerol with carboxylic acids, fatty acids and metal salts thereof, ketosulfones or mixtures of the specified compounds.

Particular preference is given to ethylene glycol distearates and/or polyethylene glycol distearates having on average 3 glycol units.

If the cosmetic, dermatological or pharmaceutical compositions according to the invention comprise one or more pearlizing compounds, these are preferably present in the compositions according to the invention in an amount of from 0.1 to 15.0% by weight and particularly preferably in an amount of from 1.0 to 10.0% by weight, based on the total weight of the respective composition according to the invention.

Fragrance and/or perfume oils which may be used are individual odorant compounds, e.g. the synthetic products of the ester, ether, aldehyde, ketone, alcohol and hydrocarbon types. Odorant compounds of the ester type are, for example, benzyl acetate, phenoxyethyl isobutyrate, p-tert-butylcyclohexyl acetate, linalyl acetate, dimethylbenzylcarbinyl acetate, phenylethyl acetate, linalyl benzoate, benzyl formate, ethylmethylphenyl glycinate, allyl cyclohexyl propionate, styrallyl propionate and benzyl salicylate. The ethers include, for example, benzyl ethyl ethers, the aldehydes include, for example, the linear alkanals having 8 to 18 carbon atoms, citral, citronellal, citronellyloxyacetaldehyde, cyclamenaldehyde, hydroxycitronellal, lilial and bourgeonal, the ketones include, for example, the ionones, alpha-isomethylionone and methyl cedryl ketone, the alcohols include anethol, citronellol, eugenol, geraniol, linalool, phenylethyl alcohol and terpineol, and the hydrocarbons include primarily the terpenes and balsams. Preference is given to using mixtures of different odorants which together produce a pleasing scent note.

Perfume oils may also comprise natural odorant mixtures, as are accessible from vegetable or animal sources, e.g. pine oil, citrus oil, jasmine oil, lily oil, rose oil or ylang-ylang oil. Essential oils of relatively low volatility, which in most cases are used as aromatic components, are also suitable as perfume oils, e.g. sage oil, chamomile oil, clove oil, melissa oil, mint oil, cinnamon leaf oil, linden blossom oil, juniperberry oil, vetiver oil, olibanum oil, galbanum oil and ladanum oil.

Suitable for use as opacifiers are polymer dispersions, in particular polyacrylate derivative, polyacrylamide derivative, poly(acrylate derivative-co-acrylamide derivative) dispersions, poly(styrene derivative-co-acrylate derivative) dispersions, saturated and unsaturated fatty alcohols.

The substances mentioned above under silicone oils and waxes may be used as silicones.

Mineral acids, in particular HCl, inorganic bases, in particular NaOH or KOH, and organic acids, in particular citric acid, may preferably be used as acids or bases for adjusting the pH.

The cosmetic, dermatological or pharmaceutical compositions according to the invention have a pH of preferably from 2 to 11, particularly preferably from 4.5 to 8.5 and especially preferably from 5.5 to 8.

The examples and applications which follow are intended to illustrate the invention in more detail, without, however, limiting it. All percentages are % by weight, unless explicitly stated otherwise.

### EXPERIMENTAL EXAMPLES

### A) Preparation of Isosorbide Dicaprylate

In a stirred 1 liter apparatus under a stream of nitrogen, 219.0 g (1.5 mol) of isosorbide and 461.4 g (3.2 mol) of caprylic acid are heated with stirring and under a stream of nitrogen to 180° C. The reaction mixture is heated at 180° C. until no more water of reaction distils off (about 28 h). The temperature is then gradually increased to 210° C. (altogether over about 30 h). The reaction has ended when a residual acid value of <2 mg KOH/g is reached. This gives a clear red-brown liquid.

Further analytical characteristics of the reaction product:
Acid value: 0.8 mg KOH/g, measured according to DIN EN ISO 2114
Hydroxyl value: 25.2 mg KOH/g, measured analogously to DIN 53240-2 according to method OHV-A
Saponification value: 54.6 mg KOH/g, measured according to DIN EN ISO 3681

For further purification, the product was distilled at a pressure of 1 mbar and a bottom temperature of from 210° C. to 240° C. This gives 251.6 g of a clear yellow liquid.

The isosorbide dicaprylate has the following composition:

| Substance | % by weight |
|---|---|
| isosorbide monocaprylate | 9.4 |
| isosorbide dicaprylate | 89.6 |
| remainder | 1 |

### B) Solubility of Butyl Methoxydibenzoylmethane

5.0% by weight of butyl methoxydibenzoylmethane were dissolved with stirring in warm isosorbide dicaprylate from preparation example A), and the mixture was cooled to 25° C. This gave a clear solution. An analogous procedure with 8.0% by weight of butyl methoxydibenzoylmethane also gave a clear solution.

When the above experiment is repeated with 5.0% by weight of butyl methoxydibenzoylmethane using dicaprylyl ether instead of isosorbide dicaprylate, a turbid solution is obtained, which indicates incomplete dissolution of the UV filter.

### C) Use Examples

Examples of cosmetic compositions according to the invention

The following cosmetic compositions are prepared using isosorbide dicaprylate from preparation example A):

### Formulation Example 1

### W/O Skin Care Milk

| | | % by |
|---|---|---|
| Phase | Ingredient | weight |
| A | amodimethicone glycerocarbamate | 2.0 |
| | cyclopentasiloxane | 5.0 |
| | paraffin oil | 3.5 |
| | apricot kernel oil | 1.0 |
| | grape seed oil | 0.5 |
| | microcrystalline wax | 0.7 |
| | stearic acid | 0.5 |
| | ethylhexyl cocoate | 7.0 |
| | butyl methoxydibenzoylmethane | 2.0 |
| B | Aristoflex ® AVC | 0.3 |
| C | water | ad 100 |
| | glycerol | 3.5 |
| | isosorbide dicaprylate | 1.0 |
| D | preservative | q.s. |

Preparation:

Oil phase A is heated to 80° C. and polymer B is stirred in. Phase C is added slowly in small portions with vigorous stirring, and the mixture is allowed to cool to room temperature. D is then added.

### Formulation Examples 2-7

### W/O Sun Protection Formulations with High Protection Factor

| | Formulation No. | | | | | |
|---|---|---|---|---|---|---|
| | 2 | 3 | 4 | 5 | 6 | 7 |
| | Amount of the respective ingredient | | | | | |
| Ingredient | [% by weight] | | | | | |
| C₁₂₋₁₅ alkyl benzoate | 8.0 | 8.0 | 8.0 | 8.0 | 8.0 | 8.0 |
| caprylic/capric triglyceride | 5.0 | 5.0 | 5.0 | 5.0 | 5.0 | 5.0 |
| octocrylene | 9.0 | — | 2.5 | 4.0 | — | — |
| ethylhexyl methoxycinnamate | 7.0 | 7.0 | 7.0 | — | 6.0 | 6.0 |
| butyl | 2.5 | — | 5.0 | — | — | — |
| methoxydibenzoylmethane | | | | | | |
| disodium phenyl | — | — | — | — | — | 3.0 |
| dibenzimidazole tetrasulfonate | | | | | | |
| ethylhexyl | — | — | — | — | 2.0 | — |
| bisisopentylbenzoxazolyl- | | | | | | |
| phenylmelamine | | | | | | |
| diethylamino hydroxybenzoyl | — | — | 2.0 | 1.0 | — | — |
| hexyl benzoate | | | | | | |
| bis ethylhexyloxyphenol | — | 3.0 | — | 2.0 | 4.0 | 3.0 |
| methoxyphenyl triazine | | | | | | |
| methylene bisbenzotriazolyl | — | 3.0 | — | — | — | 2.0 |
| tetramethylbutylphenol | | | | | | |
| ethylhexyl triazone | — | — | — | 3.0 | — | — |
| diethylhexyl butamido | — | — | — | — | 2.0 | — |
| triazone | | | | | | |
| polysilicone-15 | — | — | 2.0 | — | — | — |
| phenylbenzimidazole sulfonic | — | — | — | 3.0 | — | — |
| acid | | | | | | |
| titanium dioxide | — | 5.0 | 3.0 | 4.0 | 5.0 | 5.0 |
| cetearyl alcohol | 1.0 | 1.0 | 1.0 | 1.0 | 1.0 | 1.0 |
| sunflower seed oil sorbitol | 2.0 | 2.0 | 2.0 | 2.0 | 2.0 | 2.0 |
| esters | | | | | | |
| isosorbide dicaprylate | 0.8 | 0.8 | 0.8 | 0.8 | 0.8 | 0.8 |
| potassium cetylphosphate | 3.0 | 3.0 | 3.0 | 3.0 | 3.0 | 3.0 |
| Aristoflex ® AVC | 1.0 | 0.6 | 0.5 | 0.9 | 1.0 | 1.0 |
| water | ad | ad | ad | ad | ad | ad |
| | 100 | 100 | 100 | 100 | 100 | 100 |
| nylon | — | 0.5 | — | — | — | — |
| bisethylhexyl | — | — | 1.0 | — | — | — |
| hydroxydimethoxy | | | | | | |
| benzylmalonate | | | | | | |
| talc | — | — | — | — | 0.5 | — |
| preservative | q.s. | q.s. | q.s. | q.s. | q.s. | q.s. |

Preparation:

For the preparation, the oil-soluble components were heated to 80° C., potassium cetylphosphate and Aristoflex® AVC were poured in and the combined water-soluble phases were slowly, with vigorous stirring, introduced into the oil phase. The emulsions formed were allowed to cool to room temperature with stirring. The preservative is then worked in with stirring.

The UV filters used in formulation examples 2-7, their trade names and their UV protection range are listed in the table below.

| | | Protection |
|---|---|---|
| | | range |
| | | (UV-A/ |
| UV filter | Trade name | UV-B) |
| octocrylene | Neo Heliopan ® 303 | B |
| ethylhexyl methoxycinnamate | Neo Heliopan ® AV | B |
| butyl methoxydibenzoylmethane | Neo Heliopan ® 357, | A |
| | Parsol ® 1789 | |
| disodium phenyl dibenzimidazole | Neo Heliopan ® AP | A |
| tetrasulfonate | | |
| ethylhexyl | Uvasorb ® K2A | A |
| bisisopentylbenzoxazolyl- | | |
| phenylmelamine | | |
| diethylamino hydroxybenzoyl | Uvinul ® A Plus | A |
| hexyl benzoate | | |
| bis ethylhexyloxyphenol | Tinosorb ® S | A/B |
| methoxyphenyl triazine | | |
| methylene bisbenzotriazolyl | Tinosorb ® M | A/B |
| tetramethylbutylphenol | | |
| ethylhexyl triazone | Uvinul ® T 150 | B |
| diethylhexyl butamido triazone | Uvasorb ® HEB | B |
| polysilicone-15 | Parsol ® SLX | B |
| phenylbenzimidazole sulfonic acid | | B |

### Formulation Example 8

### O/W Sun Protection Cream

| | | % by |
|---|---|---|
| Phase | Ingredient | weight |
| A | ethylhexyl methoxycinnamate | 6.0 |
| | ethylhexyltriazone | 2.0 |
| | benzophenone-3 | 2.0 |
| | BHT | 0.05 |
| B | Aristoflex ® AVS | 1.5 |
| | trilaureth-4 phosphate | 2.0 |
| | polyglyceryl-2 sesquiisostearate | 1.0 |
| | caprylyl methicone | 1.0 |
| | isosorbide dicaprylate | 1.3 |
| | PVP/hexadecene copolymer | 1.0 |
| | tocopheryl acetate | 0.5 |
| | fragrance | 0.2 |
| C | water | ad 100 |
| | disodium EDTA | 0.1 |
| D | methylene bisbenzotriazolyl tetramethylbutylphenol | 4.0 |
| E | triethanolamine | q.s. |
| F | preservative | q.s. |

### Preparation:

Phase A is homogenized and dissolved at 60° C. and stirred into phase B, phase C is then added with stirring and the mixture is stirred at 300 revolutions per minute. Phase D is then stirred in, and the pH is adjusted to 6.8-7.2 using E. The preservative is then added with stirring.

1. A cosmetic, dermatological or pharmaceutical composition, comprising
a) one or more compounds of the formula (I) 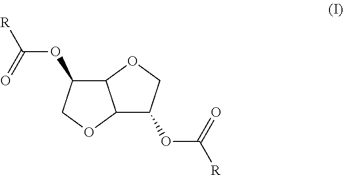
in which
R is a straight-chain or branched saturated alkyl group having 5 to 11 carbon atoms or a straight-chain or branched mono- or polyunsaturated alkenyl group having 5 to 11 carbon atoms, and
b) one or more organic or inorganic UV filters, where the composition comprises, in addition to the one or more compounds of the formula (I), one or more compounds selected from the group consisting of sorbitol, sorbitol esters, sorbitan, sorbitan esters, isosorbide, isosorbide monoesters and carboxylic acids, where the carboxylic acids themselves and also the carboxylic acids on which the acid components of the esters mentioned are based correspond to the formula RCOOH in which R has the meaning given for formula (I), and the hydroxyl value of the mixture of the one or more compounds of the formula (I) and the one or more compounds selected from the group consisting of sorbitol, sorbitol esters, sorbitan, sorbitan esters, isosorbide, isosorbide monoesters and carboxylic acids is smaller than or equal to 250.

2. The composition as claimed in claim 1, wherein the radical R in formula (I) is a straight-chain saturated alkyl radical having 7 to 9 carbon atoms.

3. The composition as claimed in claim 2, wherein the radical R in formula (I) is a straight-chain saturated alkyl radical having 7 carbon atoms.

4. The composition as claimed in claim 1, wherein the one or more UV filters of component b) are selected from organic UV filters.

5. The composition as claimed in claim 4, wherein the one or more organic UV filters of component b) are selected from the group consisting of butyl methoxydibenzoylmethane (BMDBM), octocrylene, ethylhexyl methoxycinnamate (EMC), ethylhexyl triazone (EHT), diethylhexyl butamido triazone, bisethylhexyloxyphenol methoxyphenyl triazine, 4-methylbenzylidene camphor, benzyl salicylate, ethylhexyl salicylate, terephthalydene dicamphor sulfonic acid, ethylhexyl methoxycinnamate and disodium octanyl dibenzimidazole tetrasulfonate.

6. The composition as claimed in claim 5, wherein the one or more organic UV filters of component b) are selected from the group consisting of butyl methoxydibenzoylmethane, octocrylene, ethylhexyl methoxycinnamate and ethylhexyl triazone.

7. The composition as claimed in claim 6, wherein the organic UV filter of component b) is butyl methoxydibenzoylmethane.

8. The composition as claimed in claim 1, which comprises one or more isosorbide monoesters of the formula (II) 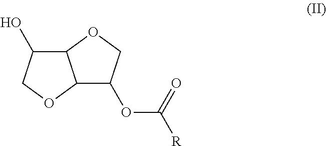
where R has the meaning given for formula (I) and where the isosorbide monoester is isosorbide monocaprylate.

9. The composition as claimed in claim 8, which comprises one or more compounds of the formula (I) and additionally II) from 0.001 to 0.2 part by weight of the one or more isosorbide monoesters of the formula (II), in each case based on 1.0 part by weight of the one or more compounds of the formula (I).

10. The composition as claimed in claim 1, which additionally comprises one or more sorbitan esters of sorbitan and carboxylic acids R^{a}COOH, where R^{a} is a straight-chain or branched saturated alkyl group having 5 to 11 carbon atoms or a straight-chain or branched mono- or polyunsaturated alkenyl group having 5 to 11 carbon atoms, and the weight ratio of the one or more compounds of the formula (I) to the one or more sorbitan esters just mentioned is from 70:30 to 100:0.

11. The composition as claimed in claim 10, wherein the one or more sorbitan esters of sorbitan and carboxylic acids R^{a}COOH are selected from sorbitan esters of sorbitan and caprylic acid.

12. The composition as claimed in claim 1, which comprises the one or more compounds of component a) in amounts of from 0.01 to 10.0% by weight and the one or more substances of component b) in amounts of from 0.01 to 40.0% by weight, in each case based on the total weight of the composition.

13. The composition as claimed in claim 1, which is formulated on an aqueous or aqueous-alcoholic basis or is present as a solution, emulsion or dispersion.

14. The composition as claimed in claim 1, which has a pH of from 2 to 11.

15. The composition as claimed in claim 4, which comprises one or more isosorbide monoesters of the formula (II) 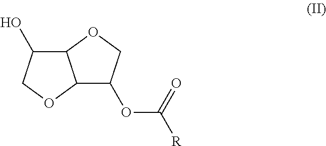
where R has the meaning given for formula (I) and where the isosorbide monoester is isosorbide monocaprylate.

16. The composition as claimed in claim 15, which comprises one or more compounds of the formula (I) and additionally
II) from 0.001 to 0.2 part by weight of the one or more isosorbide monoesters of the formula (II),
in each case based on 1.0 part by weight of the one or more compounds of the formula (I).

17. The composition as claimed in claim 15, which additionally comprises one or more sorbitan esters of sorbitan and carboxylic acids R^{a}COOH, where R^{a} is a straight-chain or branched saturated alkyl group having 5 to 11 carbon atoms or a straight-chain or branched mono- or polyunsaturated alkenyl group having 5 to 11 carbon atoms, and the weight ratio of the one or more compounds of the formula (I) to the one or more sorbitan esters just mentioned is from 70:30 to 100:0.

18. The composition as claimed in claim 4, which additionally comprises one or more sorbitan esters of sorbitan and carboxylic acids R^{a}COOH, where R^{a} is a straight-chain or branched saturated alkyl group having 5 to 11 carbon atoms or a straight-chain or branched mono- or polyunsaturated alkenyl group having 5 to 11 carbon atoms, and the weight ratio of the one or more compounds of the formula (I) to the one or more sorbitan esters just mentioned is from 70:30 to 100:0.
